(19) United States
(12) Patent Application Publication
(10) Pub. No.: US 2025/0268640 A1
(43) Pub. Date: Aug. 28, 2025

(54) SYSTEMS AND METHODS FOR PULSED FIELD ABLATION WITH SUPERIMPOSED WAVEFORMS

(71) Applicant: St. Jude Medical, Cardiology Division, Inc., St. Paul, MN (US)

(72) Inventors: Derek C. Sutermeister, Ham Lake, MN (US); Troy T. Tegg, Elk River, MN (US); Timothy S. Marass, Minneapolis, MN (US); Lori Bailey, Chatfield, MN (US); Lakshya Mittal, Dayton, MN (US); Isaac Remer, Richfield, MN (US)

(21) Appl. No.: 19/058,881

(22) Filed: Feb. 20, 2025

Related U.S. Application Data
(60) Provisional application No. 63/556,591, filed on Feb. 22, 2024.

(51) Int. Cl. A61B 18/00

(52) U.S. Cl. CPC A61B 18/00; A61B 2018/0016; A61B 2018/0022; A61B 2018/00267; A61B 2018/00613; A61B 2018/0075; A61B 2018/00761

(57) ABSTRACT

An electroporation system is provided. The system includes a catheter including a plurality of electrodes, and a pulse generator coupled to the catheter. The pulse generator is configured to generate a first waveform to be delivered between at least one active electrode on the catheter and at least one first return electrode, generate a second waveform to be delivered between the at least one active electrode and at least one second return electrode, and cause delivery of the first and second waveforms over the same time period such that at least the first and second waveforms are superimposed onto one another to create a combined waveform at the at least one active electrode.

### CROSS REFERENCE TO RELATED APPLICATIONS

This application claims priority to U.S. Provisional Patent Application No. 63/556,591 filed on Feb. 22, 2024, which is incorporated by reference herein in its entirety.

### FIELD OF THE DISCLOSURE

The present disclosure relates generally to tissue ablation systems. In particular, the present disclosure relates to systems and methods for pulsed field ablation that use superimposed waveforms.

### BACKGROUND

It is generally known that ablation therapy may be used to treat various conditions afflicting the human anatomy. For example, ablation therapy may be used in the treatment of atrial arrhythmias. When tissue is ablated, or at least subjected to ablative energy generated by an ablation generator and delivered by an ablation catheter, lesions form in the tissue. Electrodes mounted on or in ablation catheters are used to cause tissue destruction in cardiac tissue to correct conditions such as ventricular and atrial arrhythmias (including, but not limited to, ectopic atrial tachycardia, atrial fibrillation, and atrial flutter).

Arrhythmia (i.e., irregular heart rhythm) can create a variety of dangerous conditions including loss of synchronous atrioventricular contractions and stasis of blood flow which can lead to a variety of ailments and even death. It is believed that the primary cause of atrial arrhythmia is stray electrical signals within the left or right atrium of the heart. The ablation catheter imparts ablative energy (e.g., radiofrequency energy, cryoablation, lasers, chemicals, high-intensity focused ultrasound, etc.) to cardiac tissue to create a lesion in the cardiac tissue. This lesion disrupts undesirable electrical pathways and thereby limits or prevents stray electrical signals that lead to arrhythmias.

Electroporation is a substantially non-thermal ablation technique that involves applying strong electric fields that induce pore formation in the cellular membrane along with cell wide damage through various mechanism to cause cell death. The electric field may be induced by applying a relatively short duration pulse which may last, for instance, from a nanosecond to several milliseconds. Such a pulse may be repeated to form a pulse train. When such an electric field is applied to tissue in an in vivo setting, the cells in the tissue are subjected to an increased trans-membrane potential, which opens the pores on the cell plasma membrane. Electroporation may be reversible (i.e., the temporally-opened pores will reseal) or irreversible (i.e., the pores will remain open). For example, in the field of gene therapy, reversible electroporation (i.e., temporarily open pores) is used to transfect high molecular weight therapeutic vectors into the cells. In other therapeutic applications, a suitably configured pulse train alone may be used to cause cell destruction, for instance by causing irreversible electroporation.

For example, pulsed field ablation (PFA) may be used to perform instantaneous pulmonary vein isolation (PVI). PFA generally involves delivering high voltage pulses from electrodes disposed on a catheter. For example, voltage pulses may range from less than about 500 volts to about 2400 volts or higher. These fields may be applied between pairs of electrodes (bipolar therapy) or between one or more electrodes and a return patch (monopolar therapy).

In PFA, different waveforms may be used to achieve different goals. For example, some waveforms may result in larger or smaller lesion size than other waveforms. Further, some waveforms result in higher or lower overall energy delivery than other waveforms (less overall energy delivery generally corresponds to less heating of the target tissue). As another example, some waveforms are more likely to induce muscular contractions in a patient. Generally, it is desirable to deliver electroporation therapy with a relatively low number of therapy applications over a relatively short timeframe. Further, it is generally desirable to avoid thermal heating of the tissue, and to have little to no skeletal muscle recruitment (i.e., avoiding muscle contractions). In addition, it is also generally desirable to reduce the likelihood of waveforms generating sustained atrial arrhythmias.

### BRIEF SUMMARY OF THE DISCLOSURE

In one aspect, an electroporation system is provided. The system includes a catheter including a plurality of electrodes, and a pulse generator coupled to the catheter. The pulse generator is configured to generate a first waveform to be delivered between at least one active electrode on the catheter and at least one first return electrode, generate a second waveform to be delivered between the at least one active electrode and at least one second return electrode, and cause delivery of the first and second waveforms over the same time period such that at least the first and second waveforms are superimposed onto one another to create a combined waveform at the at least one active electrode.

In another aspect, a pulse generator for use with an electroporation system is provided. The pulse generator is configured to be coupled to a catheter including a plurality of electrodes. The pulse generator is configured to generate a first waveform to be delivered between at least one active electrode on the catheter and at least one first return electrode, generate a second waveform to be delivered between the at least one active electrode and at least one second return electrode, and cause delivery of the first and second waveforms over the same time period such that at least the first and second waveforms are superimposed onto one another to create a combined waveform at the at least one active electrode.

In yet another aspect, a method for electroporation therapy is provided. The method includes generating, using a pulse generator, a first waveform to be delivered between at least one active electrode on a catheter and at least one first return electrode, generating a second waveform to be delivered between the at least one active electrode and at least one second return electrode, and causing delivery of the first and second waveforms over the same time period such that at least the first and second waveforms are superimposed onto one another to create a combined waveform at the at least one active electrode.

The foregoing and other aspects, features, details, utilities, and advantages of the present disclosure will be apparent from reading the following description and claims, and from reviewing the accompanying drawings.

### BRIEF DESCRIPTION OF THE DRAWINGS

FIG. 1 is a schematic and block diagram view of a system for electroporation therapy.

FIGS. 2A and 2B are views of one embodiment of a catheter assembly that may be used with the system shown in FIG. 1.

FIGS. 3A-3C are views of alternative embodiments of a catheter assembly that may be used with the system shown in FIG. 1.

FIG. 4 is a schematic view of an alternative embodiment of a catheter assembly that may be used with the system shown in FIG. 1.

FIG. 5 is a schematic view of an alternative embodiment of a catheter assembly that may be used with the system shown in FIG. 1.

FIG. 6 is one embodiment of a waveform that may be delivered using the electroporation generator shown in FIG. 1.

FIG. 7 is one embodiment of overlaying multiple waveforms onto one another.

FIG. 8 is another embodiment of overlaying multiple waveforms onto one another.

FIG. 9 is another embodiment of overlaying multiple waveforms onto one another.

FIG. 10 is another embodiment of overlaying multiple waveforms onto one another.

FIG. 11 is another embodiment of overlaying multiple waveforms onto one another.

FIG. 12 is another embodiment of overlaying multiple waveforms onto one another.

FIG. 13 is a comparison of a first applied waveform and a second applied waveform.

### DETAILED DESCRIPTION OF THE DISCLOSURE

The present disclosure provides systems and methods for electroporation.

An electroporation system includes a catheter including a plurality of electrodes, and a pulse generator coupled to the catheter. The pulse generator is configured to generate a first waveform to be delivered between at least one active electrode on the catheter and at least one first return electrode, generate a second waveform to be delivered between the at least one active electrode and at least one second return electrode, and cause delivery of the first and second waveforms over the same time period such that at least the first and second waveforms are superimposed onto one another to create a combined waveform at the at least one active electrode.

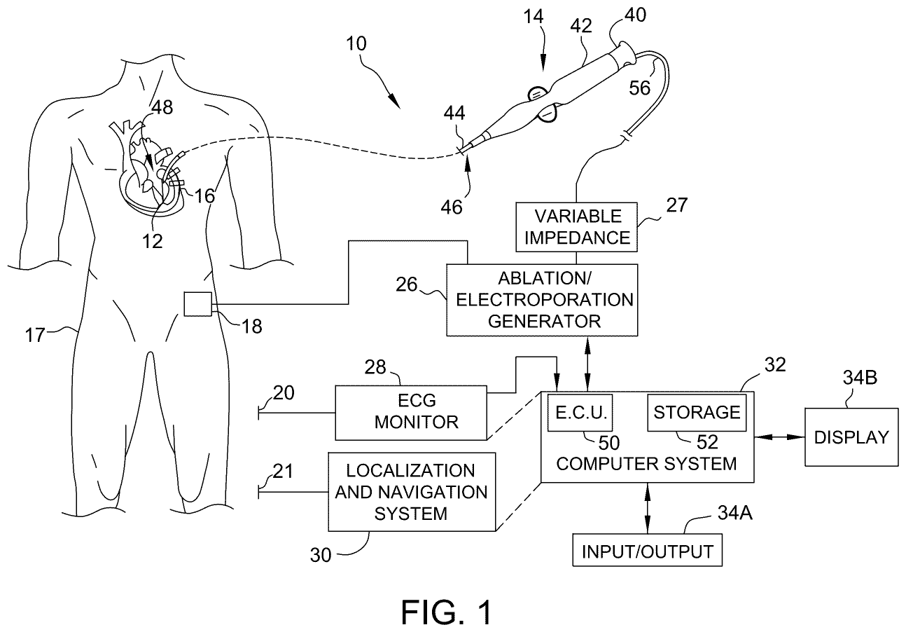

FIG. 1 is a schematic and block diagram view of a system 10 for electroporation therapy. In general, system 10 includes a catheter electrode assembly 12 disposed at a distal end 48 of a catheter 14. As used herein, “proximal” refers to a direction toward the end of the catheter 14 near the clinician and “distal” refers to a direction away from the clinician and (generally) inside the body of a patient. The electrode assembly 12 includes one or more individual, electrically-isolated electrode elements. Each electrode element, also referred to herein as a catheter electrode, is individually wired such that it can be selectively paired or combined with any other electrode element to act as a bipolar or a multi-polar electrode.

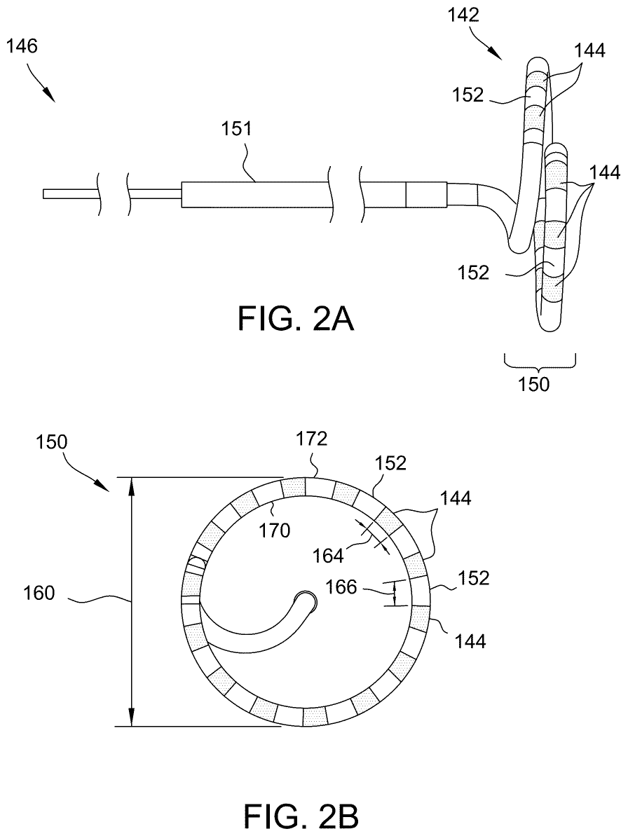

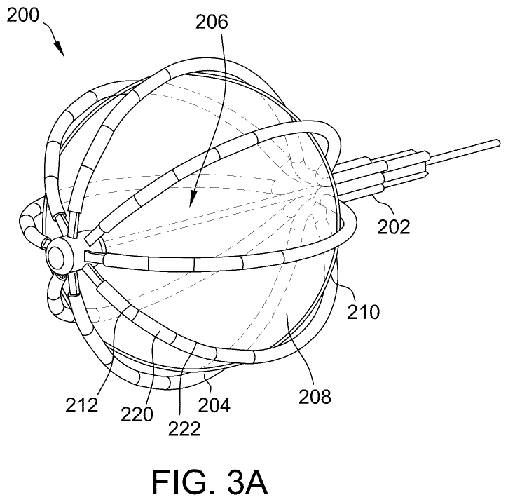

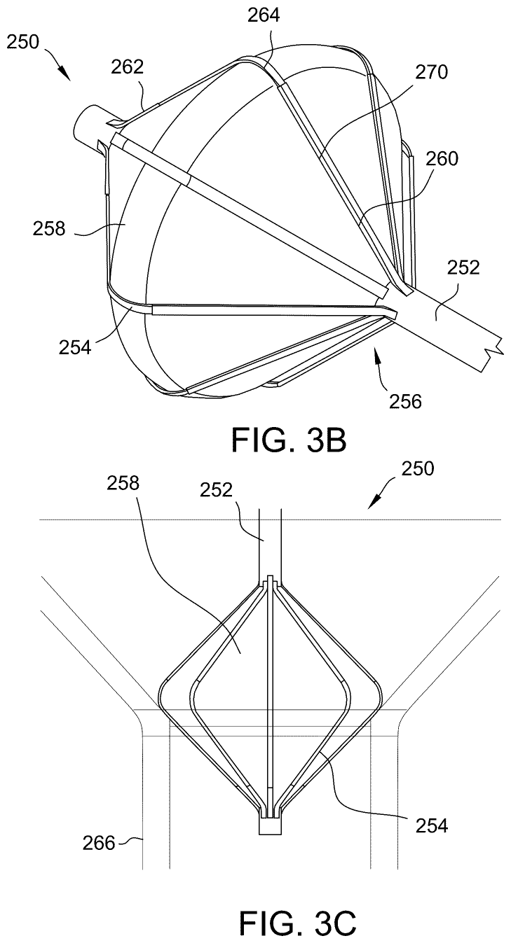

System 10 may be used for irreversible electroporation (IRE) to destroy tissue. In particular, system 10 may be used for electroporation-induced therapy that includes delivering electrical pulses in such a manner as to directly cause an irreversible loss of plasma membrane integrity leading to its breakdown and cell destruction. This mechanism of cell destruction may be viewed as an “outside-in” process, meaning that the disruption of the outside plasma membrane of the cell causes detrimental effects to the inside of the cell. Sometimes these electrical pulses may directly manipulate and damage the intracellular organelles to induce cell death, without causing a significant amount of damage to the cell membrane. Typically, for classical electroporation, electric energy may be delivered as a pulsed electric field in the form of short-duration pulses (e.g., having a 10 nanosecond (ns) to 100 millisecond (ms) duration) between closely spaced electrodes capable of delivering an electric field strength of about 0.05 to 100.0 kilovolts/centimeter (kV/cm). System 10 may be used for high output (e.g., high voltage and/or high current) electroporation procedures. Further, system 10 may be used with a loop catheter such as that depicted in FIGS. 2A and 2B, and/or with a basket catheter such as those depicted in FIGS. 3A-3C. In some embodiments, system 10 is used for reversible electroporation instead of or in addition to irreversible electroporation.

In one embodiment, stimulation is delivered selectively (e.g., between pairs of electrodes) on catheter 14. Further, the electrodes on catheter 14 may be switchable between being connected to a 3D mapping system and being connected to an electroporation generator.

Irreversible electroporation through a multi-electrode catheter may enable pulmonary vein isolation in as few as one shock per vein, which may produce much shorter procedure times compared to sequentially positioning a radiofrequency (RF) ablation tip around a vein. Further, irreversible electroporation may be used for focal ablation procedures. Notably, the embodiments described herein may be used with any suitable irreversible electroporation application.

It should be understood that while the energization strategies are described as involving square wave pulses, embodiments may use variations and remain within the spirit and scope of the disclosure. For example, exponentially-decaying pulses, exponentially-increasing pulses, and combinations may be used.

Further, it should be understood that the mechanism of cell destruction in electroporation is not primarily due to heating effects, but rather to cell membrane disruption (e.g., through pore formation and/or other cell damage), damage to intracellular organelles and oxidative stress, etc., causing a cell wide effect through application of a high-voltage electric field. Thus, electroporation may avoid some possible thermal effects that may occur when using radio frequency (RF) energy. This “colder therapy” thus has desirable characteristics.

With this background, and now referring again to FIG. 1, system 10 includes a catheter electrode assembly 12 including at least one catheter electrode. Electrode assembly 12 is incorporated as part of a medical device such as a catheter 14 for electroporation therapy of tissue 16 in a body 17 of a patient. In the illustrative embodiment, tissue 16 includes heart or cardiac tissue. It should be understood, however, that embodiments may be used to conduct electroporation therapy with respect to a variety of other body tissues (e.g., renal tissue, tumors, etc.).

FIG. 1 further shows a plurality of return electrodes designated 18, 20, and 21, which are diagrammatic of the body connections that may be used by the various sub-systems included in overall system 10, such as an electroporation generator 26, an electrophysiology (EP) monitor such as an ECG monitor 28, and a localization and navigation system 30 for visualization, mapping, and navigation of internal body structures. In the illustrated embodiment, return electrodes 18, 20, and 21 are patch electrodes. It should be understood that the illustration of a single patch electrode is diagrammatic only (for clarity) and that such sub-systems to which these patch electrodes are connected may, and typically will, include more than one patch (body surface) electrode, and may include split patch electrodes (as described herein). Further, in some embodiments, in a multiplexing arrangement, therapy may repeatedly switch between using a different return electrodes 18, 20, 21. In other embodiments, return electrodes 18, 20, and 21 may be any other type of electrode suitable for use as a return electrode including, for example, one or more catheter electrodes. Return electrodes that are catheter electrodes may be part of electrode assembly 12 or part of a separate catheter or device (not shown). System 10 may further include a main computer system 32 (including an electronic control unit 50 and data storage-memory 52), which may be integrated with localization and navigation system 30 in certain embodiments. System 32 may further include conventional interface components, such as various user input/output mechanisms 34A and a display 34B, among other components.

Electroporation generator 26 is configured to energize the electrode element(s) in accordance with an electroporation energization strategy, which may be predetermined or may be user-selectable. For electroporation therapy, generator 26 may be configured to produce an electric energy that is delivered via electrode assembly 12 as a pulsed electric field in the form of short-duration square wave pulses (e.g., a nanosecond to several milliseconds duration, or any duration suitable for electroporation) between closely spaced electrodes capable of delivering an electric field strength (i.e., at the tissue site) of about 0.05 to 100.0 kV/cm. The amplitude and pulse width needed for irreversible electroporation are nearly inversely related. That is, as pulse widths are decreased, the amplitude may generally be increased to achieve chronaxie. The electric energy may be delivered, for example, using a fixed voltage delivery system (in which a fixed voltage is applied, independent of a patient impedance) or using a fixed current delivery system (in which a fixed current is achieved by adjusting the voltage based on the patient impedance). In a fixed current delivery system, the patient impedance may be determined, for example, by delivering a relatively small voltage pulse and measuring current to calculate impedance, or by delivering an AC current waveform and measuring voltage to calculate impedance.

Electroporation generator 26, sometimes also referred to herein as a DC energy source, is a biphasic electroporation generator 26 configured to generate a series of energy pulses that all produce current in two directions (i.e., positive and negative pulses). In other embodiments, electroporation generator 26 is a monophasic or polyphasic electroporation generator 26. In some embodiments, electroporation generator 26 is configured to output energy in pulses at selectable energy levels, such as fifty joules, one hundred joules, two hundred joules, and the like. Other embodiments may have more, or fewer energy settings and the values of the available setting may be the same or different (settings may include, e.g., waveform parameters, voltage, current, number of applications, etc.). For successful electroporation, some embodiments utilize the two hundred joule output level. For example, electroporation generator 26 may output a pulse having a peak magnitude from about 10 Volts (V) to about 10,200 V. Other embodiments may output any other suitable positive or negative voltage.

In some embodiments, a variable impedance 27 allows the impedance of system 10 to be varied to limit arcing. Moreover, variable impedance 27 may be used to change one or more characteristics, such as amplitude, duration, pulse shape, and the like, of an output of electroporation generator 26. Although illustrated as a separate component, variable impedance 27 may be incorporated in catheter 14 or generator 26.

With continued reference to FIG. 1, as noted above, catheter 14 may include functionality for electroporation and in certain embodiments also additional ablation functions (e.g., RF ablation). It should be understood, however, that in those embodiments, variations are possible as to the type of ablation energy provided (e.g., cryoablation, ultrasound, etc.).

In the illustrative embodiment, catheter 14 includes a cable connector or interface 40, a handle 42, and a shaft 44 having a proximal end 46 and a distal end 48. Catheter 14 may also include other conventional components not illustrated herein such as a temperature sensor, additional electrodes, and corresponding conductors or leads. Connector 40 provides mechanical and electrical connection(s) for cable 56 extending from generator 26. Connector 40 may include conventional components known in the art and as shown is disposed at the proximal end of catheter 14.

Handle 42 provides a location for the clinician to hold catheter 14 and may further provide means for steering or the guiding shaft 44 within body 17. For example, handle 42 may include means to change the length of a guidewire extending through catheter 14 to distal end 48 of shaft 44 or means to steer shaft 44. Moreover, in some embodiments, handle 42 may be configured to vary the shape, size, and/or orientation of a portion of the catheter, and it will be understood that the construction of handle 42 may vary. In an alternate embodiment, catheter 14 may be robotically driven or controlled. Accordingly, rather than a clinician manipulating a handle to advance/retract and/or steer or guide catheter 14 (and shaft 44 thereof in particular), a robot is used to manipulate catheter 14. Shaft 44 is an elongated, tubular, flexible member configured for movement within body 17. Shaft 44 is configured to support electrode assembly 12 as well as contain associated conductors, and possibly additional electronics used for signal processing or conditioning. Shaft 44 may also permit transport, delivery and/or removal of fluids (including irrigation fluids and bodily fluids), medicines, biologics, and/or surgical tools or instruments. Shaft 44 may be made from conventional materials such as polyurethane and defines one or more lumens configured to house and/or transport electrical conductors, fluids or surgical tools, as described herein. Shaft 44 may be introduced into a blood vessel or other structure within body 17 through a conventional introducer. Shaft 44 may then be advanced/retracted and/or steered or guided through body 17 to a desired location such as the site of tissue 16, including through the use of guidewires or other means known in the art.

Localization and navigation system 30 may be provided for visualization, mapping and navigation of internal body structures. Localization and navigation system 30 may include conventional apparatus known generally in the art. For example, localization and navigation system 30 may be substantially similar to the EnSite Precision™ System, commercially available from Abbott Laboratories, and as generally shown in commonly assigned U.S. Pat. No. 7,263,397 titled “Method and Apparatus for Catheter Navigation and Location and Mapping in the Heart”, the entire disclosure of which is incorporated herein by reference. In another example, localization and navigation system 30 may be substantially similar to the EnSite X™ System, as generally shown in U.S. Pat. App. Pub. No. 2020/0138334 titled “Method for Medical Device Localization Based on Magnetic and Impedance Sensors”, the entire disclosure of which is incorporated herein by reference. It should be understood, however, that localization and navigation system 30 is an example only, and is not limiting in nature. Other technologies for locating/navigating a catheter in space (and for visualization) are known, including for example, the CARTO navigation and location system of Biosense Webster, Inc., the Rhythmia® system of Boston Scientific Scimed, Inc., the KODEX® system of Koninklijke Philips N. V., the AURORA® system of Northern Digital Inc., commonly available fluoroscopy systems, or a magnetic location system such as the gMPS system from Mediguide Ltd.

In this regard, some of the localization, navigation and/or visualization systems may include a sensor for producing signals indicative of catheter location information, and may include, for example, one or more electrodes in the case of an impedance-based localization system, or alternatively, one or more coils (i.e., wire windings) configured to detect one or more characteristics of a magnetic field, for example in the case of a magnetic-field based localization system. As yet another example, system 10 may utilize a combination electric field-based and magnetic field-based system as generally shown with reference to U.S. Pat. No. 7,536,218 entitled “Hybrid Magnetic-Based and Impedance Based Position Sensing,” the disclosure of which is incorporated herein by reference in its entirety.

Pulsed field ablation (PFA), which is a methodology for achieving irreversible electroporation and cell death, may be implemented using the systems and methods described herein. In some cases, PFA may be used at specific cardiac tissue sites such as the pulmonary veins to perform a pulmonary vein isolation (PVI), or to perform focal ablation. In PFA, electric fields may be applied between adjacent electrodes (in a bipolar approach), or between one or more catheter electrodes and a return patch (in a monopolar approach), or between a combination of catheter electrodes and a return patch (in a multipolar approach). There are advantages and disadvantages to each of these approaches.

Both approaches, using an appropriate electrode geometry, are able to provide contiguous lesions. For lesion size and proximity, the monopolar approach can potentially create deeper lesions with the same applied voltage. Further, the monopolar approach may be able to create lesions from a distance (e.g., generally proximate, but not necessarily contacting tissue). The bipolar approach may create smaller lesions, requiring closer proximity or contact with tissue to create transmural lesions (depending on, for example, tissue thickness). To monitor operation of system 10, one or more impedances between catheter electrodes 144 and/or return electrodes 18, 20, and 21 may be measured. For example, for system 10, impedances may be measured as described in U.S. Patent Application Publication No. 2019/0117113, filed on Oct. 23, 2018, U.S. Patent Application Publication No. 2019/0183378, filed on Dec. 19, 2018, and U.S. Patent Application No. 63/027,660, filed on May 20, 2020, all of which are incorporated by reference herein in their entirety.

FIGS. 2A and 2B are views of one embodiment of a catheter assembly 146 that may be used with catheter 14 in system 10. Catheter assembly 146 may be referred to as a loop catheter.

Those of skill in the art will appreciate that, in other embodiments, any suitable catheter may be used. That is, the systems and methods described herein are not limited to use with the particular catheter assemblies shown. For example, the systems and methods described herein may be implemented in a linear catheter, a grid catheter (e.g., a catheter including a number of splines arranged in a plane, each spline including one or more electrodes), a balloon catheter, a sheath, a sleeve, and/or a focal ablation catheter (e.g., such as the Abbott TactiFlex catheter).

Specifically, FIG. 2A is a side view of catheter assembly 146 with a variable diameter loop 150 at a distal end 142. FIG. 2B is an end view of variable diameter loop 150 of catheter assembly 146. Those of skill in the art will appreciate that the methods and systems described herein may be implemented using any suitable catheter (e.g., fixed loop catheters, linear catheters, basket catheter, etc.). As shown in FIGS. 2A and 2B, variable diameter loop 150 is coupled to a distal section 151 of shaft 44.

Variable diameter loop 150 is selectively transitionable between an expanded (also referred to as “open”) diameter 160 (shown in FIG. 2A) and a retracted (also referred to as “closed”) diameter 160 (not shown). In the example embodiment, an expanded diameter 160 is twenty eight mm and a retracted diameter 160 is fifteen mm. In other embodiments, diameter 160 may be variable between any suitable open and closed diameters 160.

In the embodiment shown, variable diameter loop 150 includes fourteen catheter electrodes 144 substantially evenly spaced around the circumference of variable diameter loop 150 in the expanded configuration. In the retracted configuration, one or more of electrodes 144 may overlap. In other embodiments, other arrangements of catheter electrodes 144 may be implemented. For example, in one embodiment, variable diameter loop 150 includes twelve catheter electrodes 144.

Catheter electrodes 144 may be, for example, platinum ring electrodes configured to conduct and/or discharge electrical current in the range of one thousand volts and/or ten amperes. In other embodiments, variable diameter loop 150 may include any suitable number of catheter electrodes 144 made of any suitable material. Catheter electrodes 144 may include any catheter electrode suitable to conduct high voltage and/or high current (e.g., in the range of one thousand volts and/or ten amperes). Each catheter electrode 144 is separated from each other catheter electrode 144 by an insulated gap 152. In the example embodiment, each catheter electrode 144 has a same length 164 (shown in FIG. 2B) and each insulated gap 152 has a same length 166 as each other gap 152. Length 164 and length 166 are both about 2.5 mm in the example embodiment. In other embodiments, length 164 and length 166 may be different from each other. Moreover, in some embodiments, catheter electrodes 144 may not all have the same length 164 and/or insulated gaps 152 may not all have the same length 166. In some embodiments, catheter electrodes 144 are not spaced evenly around the circumference of variable diameter loop 150.

Diameter 160 and catheter electrode 144 spacing may be developed to provide a targeted range of energy density to tissue, as well as to provide sufficient electroporation coverage for different human anatomic geometries. In general, a sufficient number of electrodes 144 with appropriate lengths 164 are desired to provide substantially even and continuous coverage around the circumference of variable diameter loop 150, while still allowing enough flexibility to allow variable diameter loop 150 to expand and contract to vary diameter 160 to the desired extremes.

As mentioned above, length 164 of catheter electrodes 144 may be varied. Increasing length 164 of catheter electrodes 144 may increase coverage of electrodes 144 around the circumference of variable diameter loop 150 while also decreasing current density (by increasing the surface area) on electrodes 144, which may help prevent arcing and thermal effects during electroporation operations. Increasing length 164 too much, however, may prevent variable diameter loop 150 from forming a smooth circular shape and may limit the closed diameter 160 of variable diameter loop 150. Additionally, too great a length 164 may increase the surface area of catheter electrodes 144 to a point that the current density applied to catheter electrodes 144 by a power source is below the minimum current density needed for successful therapy. Conversely, decreasing length 164 decreases the surface area, thereby increasing the current density (assuming no other system changes) on catheter electrodes 144. As discussed above, greater current densities may lead to increased risk of arcing and heating during electroporation, and may result in larger additional system resistances needing to be added to prevent arcing. Moreover, in order to get a desired, even coverage about the circumference of variable diameter loop 150, more catheter electrodes 144 may be needed if length 164 is decreased. Increasing the number of catheter electrodes 144 on variable diameter loop 150 may prevent variable diameter loop 150 from being able to be contracted to a desired minimum diameter 160.

FIG. 3A is a perspective view of an alternative catheter assembly 200 that may be used with catheter 14. Catheter assembly 200 may be referred to as a basket catheter. Catheter assembly 200 includes a shaft 202 and a plurality of splines 204 surrounding a distal portion 206 of shaft 202. In this embodiment, catheter assembly 200 also includes a balloon 208 enclosed by splines 204. Balloon 208 may be selectively inflated to fill the space between splines 204. Notably, balloon 208 functions as an insulator, and generally reduces energy losses, which may result in increased lesion size. In some embodiments, balloon 208 may be filled with a cold media (e.g., a cryofluid or cold saline). Further, in some embodiments balloon 208 may be a double-layered balloon.

Each spline 204 includes a proximal end 210 coupled to shaft 202 and a distal end 212 coupled to shaft 202. From proximal end 210 to distal end 212, spline 204 has an arcuate shape that extends radially outward.

In this embodiment, each spline 204 includes a plurality of individual electrodes 220. For example, each spline 204 may include an elastic material (e.g., Nitinol) covered in a polymer tube 222, with individual electrodes 220 attached to an exterior of polymer tube 222. In the embodiment shown, each spline 204 includes two electrodes 220. Further, as shown in FIG. 2, electrodes 220 are generally positioned closer to distal end 212 than proximal end 210 to correspond to portions of spline 204 that will contact the pulmonary vein.

Alternatively, each spline 204 may include any suitable number and arrangement of electrodes 220. For example, in some embodiments, each spline 204 includes four electrodes 220.

In this embodiment, alternating splines 204 alternate polarities. That is, electrodes 220 on a particular spline 204 have the same polarity, but electrodes 220 on a particular spline 204 have a different polarity than electrodes 220 on adjacent splines 204. Alternatively, any suitable polarization scheme may be used. During delivery, splines 204 may be collapsed in towards shaft 202. Subsequently, to perform ablation, splines 204 are deployed to extend radially outward.

Splines 204 may all have the same length, or at least some of splines 204 may have different lengths. Further, insulating material on each spline 204 may have the same length, or at least some splines 204 may have insulating material with different lengths. In addition, in some embodiments, catheter assembly 200 includes a distal electrode (not shown) positioned distal of splines 204. The distal electrode may be used to perform point ablation (e.g., by creating a bipole between the distal electrode and one of splines 204), and/or may be used for visualization/mapping purposes (e.g., using the distal electrode in combination with an electrode on shaft 202).

FIG. 3B is a perspective view of an alternative catheter assembly 250 that may be used with catheter 14, and FIG. 3C is a side schematic view of catheter assembly 250. Like catheter assembly 200 (shown in FIG. 3A), catheter assembly 250 may be referred to as a basket assembly.

Catheter assembly 250 includes a shaft 252 and a plurality of splines 254 surrounding a distal portion 256 of shaft 252. In this embodiment, catheter assembly 250 includes a balloon 258 enclosed by splines 254. Balloon 258 may be selectively inflated to occupy the space between splines 254. Notably, balloon 258 functions as an insulator, and generally reduces energy, which may result in increased lesion size.

Each spline 254 includes a proximal end 260 coupled to shaft 252 and a distal end 262 coupled to shaft 252. From proximal end 260, spline 1004 extends radially outward to an inflection point 264, and then extends radially inward to distal end 262. FIG. 3C shows catheter assembly 250 positioned within the pulmonary vein 266.

A body of each spline 254 is made of an elastic material (e.g., Nitinol), and functions as a relatively large electrode. In this embodiment, alternating splines 254 alternate polarities. That is, each positive spline 254 is positioned between two negative splines 254 and vice-versa. Alternatively, any suitable polarization scheme may be used.

To control the ablation zone of each spline 254, portions of each spline 254 may be covered with insulating material 270 (e.g., heat-shrink or polymer tubing or spray or dip coat with polyimide or PEBAX), and the exposed portions of splines 254 function as electrodes. In the embodiment shown in FIGS. 3B and 3C, inflection point 264 and portions of spline 254 between inflection point 264 and distal end 262 are generally exposed, while portions of spline 254 between inflection point 264 and proximal end 260 are generally insulated. This results in the portions of spline 254 that contact pulmonary vein 266 being exposed (see FIG. 3C). Alternatively, any suitable insulation configuration may be used.

During delivery, splines 254 and balloon 258 may be collapsed. To perform ablation, splines 254 are deployed with inflection points 264 extending radially outward, and balloon 258 is selectively inflated to occupy the space between splines 254.

The combination of balloon 258 and splines 254 facilitates straightforward delivery and deployment of catheter assembly 250. Further, balloon 258 drives more energy into ablated tissue, and stabilizes splines 254 to prevent lateral movement. In addition, using splines 254 as electrodes instead of individual smaller electrodes may facilitate reducing the cost and increasing the reliability of catheter assembly 250.

Splines 254 may all have the same length, or at least some of splines 254 may have different lengths. Further, insulating material 270 on each spline 254 may have the same length, or at least some splines 254 may have insulating material 270 with different lengths. In addition, in some embodiments, catheter assembly 250 includes a distal electrode (not shown) positioned distal of splines 254. The distal electrode may be used to perform point ablation (e.g., by creating a bipole between the distal electrode and one of splines 254), and/or may be used for visualization/mapping purposes (e.g., using the distal electrode in combination with an electrode on shaft 252).

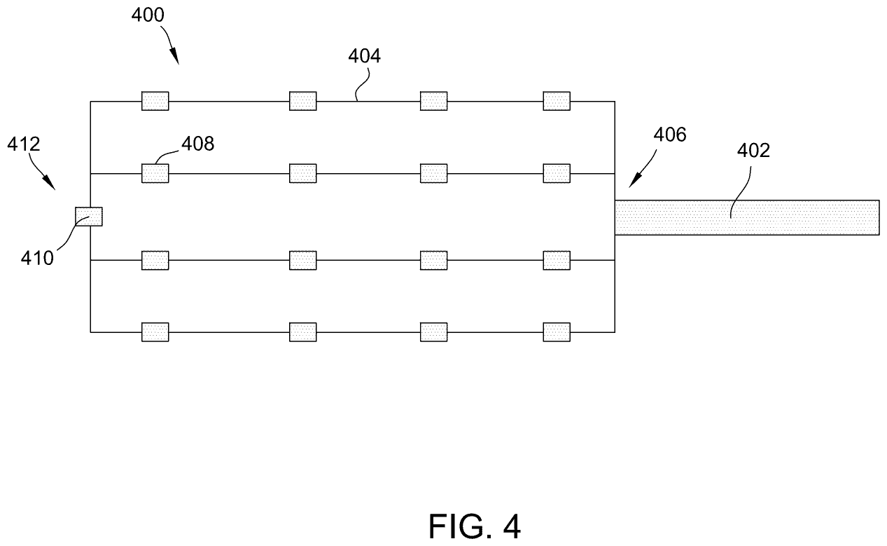

FIG. 4 is a schematic view of an alternative catheter assembly 400 that may be used with catheter 14. Catheter assembly 400 may be referred to as a grid assembly.

Catheter assembly 400 includes a shaft 402 and a plurality of parallel splines 404 arranged in a grid at a distal portion 406 of shaft 402. Each spline 404 includes one or more spline electrodes 408. Further, in this embodiment, catheter assembly 400 includes at least one tip electrode 410 at a distal end 412 of catheter assembly 400.

In the embodiment shown in FIG. 4, catheter assembly 400 includes sixteen spline electrodes 408 and one tip electrode 410. Alternatively, catheter assembly 400 may include any suitable number of electrodes.

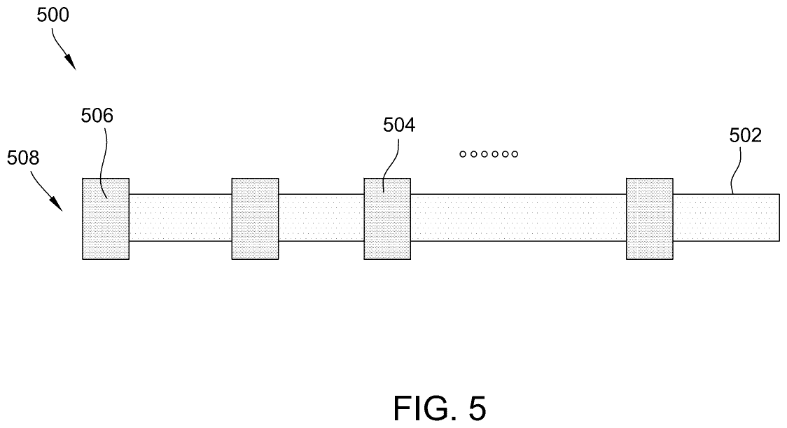

FIG. 5 is a schematic view of an alternative catheter assembly 500 that may be used with catheter 14. Catheter assembly 500 may be referred to as a linear assembly.

Catheter assembly 500 includes a shaft 502. One or more shaft electrodes 504 are arranged on shaft 502, and at least one tip electrode 506 is arranged at a distal end 508 of shaft 502. In the embodiment shown in FIG. 5, catheter assembly 500 includes at least four shaft electrodes 504 and one tip electrode 506. Alternatively, catheter assembly 500 may include any suitable number of electrodes.

Those of skill the art will appreciate that catheter assembly 146 (shown in FIG. 2A and 2B), catheter assembly 200 (shown in FIG. 3A), catheter assembly 250 (shown in FIGS. 3B and 3C), catheter assembly 400 (shown in FIG. 4), and catheter assembly 500 (shown in FIG. 5) are merely examples. Notably, the systems and methods described herein may be implemented using any suitable catheter assembly. Further, although some of the embodiments discussed herein are described in the context of a particular catheter assembly, those of skill in the art will appreciate that the systems and methods described herein may be used with any suitable catheter assembly.

For electroporation therapy, waveforms are generated using a pulse generator (e.g., electroporation generator 26 (shown in FIG. 1)) and applied between two or more catheter electrodes (i.e., a bipolar approach) or between individual catheter electrodes and a return patch (i.e., a monopolar approach). The waveforms may be monophasic, biphasic (i.e., having both a positive pulse and a negative pulse), or polyphasic. Further, the waveforms may include one or more bursts of pulses (with each burst including multiple pulses). Further, the waveforms are defined by multiple parameters (e.g., pulse width, pulse amplitude, frequency, etc.).

Different waveforms may be used to achieve different goals. For example, some waveforms may result in larger or smaller lesion size than other waveforms. Further, some waveforms result or higher or lower overall energy delivery than other waveforms (less overall energy delivery generally corresponds to less heating of the target tissue). As another example, some waveforms are more likely to induce muscular contractions in a patient. Generally, it is desirable to deliver electroporation therapy with a relatively low number of therapy applications over a relatively short timeframe. Further, it is generally desirable to avoid thermal heating of the tissue, and to have little to no skeletal muscle recruitment (i.e., avoiding muscle contractions).

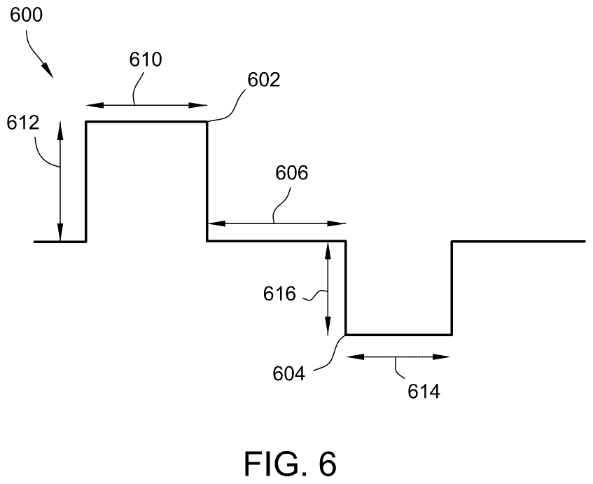

FIG. 6 is one embodiment of a waveform 600 that may be delivered using electroporation generator (shown in FIG. 1). Waveform 600 includes a positive pulse 602 followed by a negative pulse 604. Further, there is an intrapulse delay 606 between the positive and negative pulses 602 and 604.

As shown in FIG. 6, positive pulse 602 has a first pulse width 610 and a first pulse amplitude 612. Similarly, negative pulse 604 has a second pulse width 614 and a second pulse amplitude 616. Waveform 600 may be symmetric (i.e., with first pulse width 610 and first pulse amplitude 612 substantially equal to second pulse width 614 and second pulse amplitude 616) or asymmetric (i.e., with at least one of first pulse width 610 and first pulse amplitude 612 different from second pulse width 614 and second pulse amplitude 616).

When first pulse amplitude 612 and second pulse amplitude 616 are both non-zero, waveform 600 is biphasic (i.e., as shown in FIG. 6). For a monophasic waveform, one of first pulse amplitude 612 and second pulse amplitude 616 is zero. For example, if first pulse amplitude 612 is zero, waveform 600 is monophasic with single negative pulse 604. If second pulse amplitude 616 is zero, waveform 600 is monophasic with single positive pulse 602.

In one biphasic example, first and second pulse widths 610 and 614 may each be 3 microseconds (3 μs), with an intrapulse delay 606 of 1 μs. This may be referred to as a 3-1-3 waveform (i.e., first pulse width 610 of 3 μs—intrapulse 606 delay of 1 μs-second pulse width 614 of 3 μs). First and second pulse amplitudes 612 and 616 may each be, for example, on the order of 1800 Volts (1800V).

In one monophasic example, first pulse width 610 is 0 μs, and second pulse width 614 is 3 μs, with an intrapulse delay 606 of 1 μs. This may be referred to as a 0-1-3 waveform (i.e., first pulse width 610 of 0 μs—intrapulse delay 606 of 1 μs—second pulse width 614 of 3 μs). Second pulse amplitude 616 may be, for example, on the order of 1800V. In another example, the intrapulse delay 606 may be 0 μs.

In the embodiments described herein, multiple waveforms are generated using a pulse generator (e.g., electroporation generator 26 (shown in FIG. 1)), and those multiple waveforms are delivered by a catheter (e.g., catheter 14 (shown in FIG. 1) over the same time period, such that the catheter delivers a combined waveform that is the result of superimposing the multiple waveforms onto one another. This may also be referred to as nesting or overlaying the multiple waveforms. By overlaying multiple waveforms on top of each other, therapy delivery may be optimized at an active electrode or set of active electrodes while minimizing lesion creation at non-intended locations and reducing patient movement. This also enables a combination of monopolar and bipolar therapies, as described herein.

The following example is described in the context of a linear catheter assembly, such as catheter assembly 500 (shown in FIG. 5). Suppose the linear catheter assembly includes a tip electrode (such as tip electrode 506) and multiple shaft electrodes (such as shaft electrodes 504). Notably, those of skill the art will appreciate that, in the embodiments described herein, the “tip electrode” could be replaced with any other suitable electrode (e.g., a shaft electrode in a linear catheter assembly or a loop catheter assembly, or a spline electrode in a basket assembly or a grid assembly), and the “shaft electrodes” could be replaced with any other suitable electrodes (e.g., a tip electrode or a spline electrode in a basket assembly or a grid assembly).

In at least some known systems, bipolar energy is delivered between i) the tip electrode and ii) a first shaft electrode, E2. Alternatively, bipolar energy may be delivered between the i) tip electrode and ii) the first shaft electrode, E2, and a second shaft electrode, E3 (i.e., E2 and E3 are set to the same voltage level). When delivering energy in this way, a relatively powerful electric field is generated at each of the tip electrode, the first shaft electrode, and the second shaft electrode (when used). Because each electrode sees the same waveform and is exposed to the same electric field, each electrode produces relatively large lesions. However, it may be beneficial to the patient in some scenarios to limit lesion generation to less than all of the electrodes.

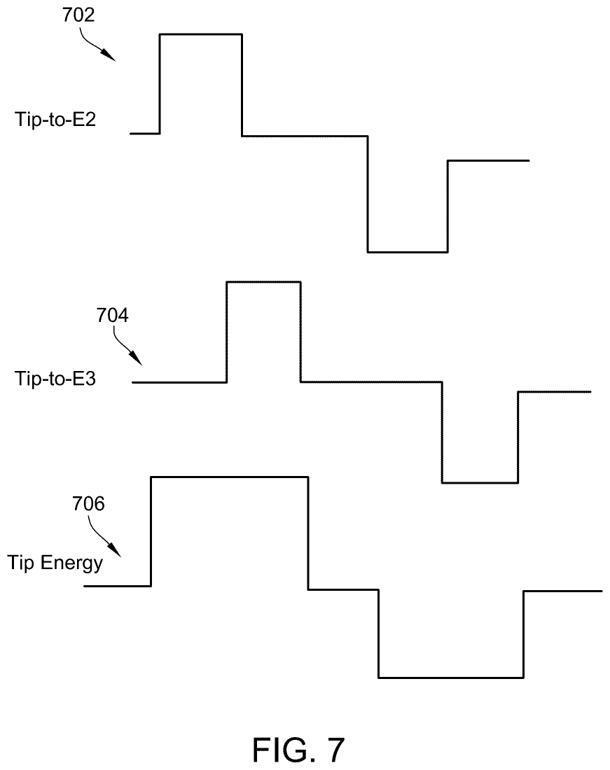

Accordingly, in one embodiment in accordance with the systems and methods described herein, a first waveform and a second waveform are overlaid onto one another, as shown in FIG. 7. Specifically, a first waveform 702 delivers energy between the tip electrode and the first shaft electrode, and a second waveform 704 delivers energy between the tip electrode and the second shaft electrode.

For example, first waveform 702 may include a 1 μs positive pulse, followed by a 2 μs delay, followed by a 1 μs negative pulse. Further, second waveform 704 may include a 1 μs delay, followed by a 1 μs positive pulse, followed by a 2 μs delay, followed by a 1 μs negative pulse. Those of skill in the art will appreciate that these values are only example values for the purposes of illustration.

Further, although in this example, first and second waveforms 702 and 704 are biphasic, those of skill in the art will appreciate that, in other embodiments at least one of first and second waveforms 702 and 704 may be monophasic. This also applies to the other examples described herein (i.e., those of skill in the art will appreciate that any suitable arrangement of biphasic and/or monophasic waveforms may be overlaid onto one another).

Referring back to FIG. 7, the first shaft electrode only sees first waveform 702, and the second shaft electrode only sees second waveform 704. However, the tip electrode sees a combined waveform 706 that is the result of superimposing first waveform 702 onto second waveform 704. Here, as shown in FIG. 7, combined waveform 706 includes a 2 μs positive pulse, followed by a 1 μs delay, followed by a 2 μs negative pulse.

This arrangement of first and second waveforms 702 and 704 results in increased lesion formation at the tip electrode, and reduced lesion formation at the first and second shaft electrodes. Accordingly, one of skill in the art will appreciate that the superimposed waveform configurations described herein facilitate maintaining lesion quality at one or more active electrodes (e.g., the tip electrode) while reducing or eliminating lesion formation at one or more ground electrodes (e.g., the first and second shaft electrodes and/or one or more external patch electrodes).

Although first and second waveforms 702 and 704 are biphasic square wave pulses in this example, those of skill in the art will appreciate that any suitable pulse shapes may be used (e.g., sinusoidal pulses, trapezoidal pulses, AC and DC pulses combined, monophasic and biphasic pulses combined, etc.).

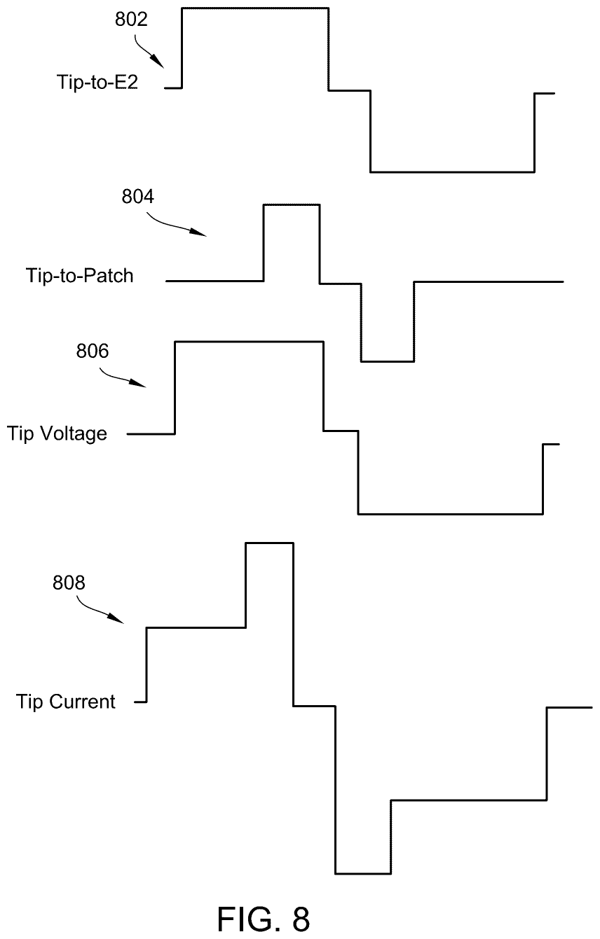

FIG. 8 illustrates another example of overlaying two waveforms. In this example, a first waveform 802 is a bipolar waveform delivered between a tip electrode and a shaft electrode, E2. In contrast, a second waveform 804 is a monopolar waveform delivered between the tip electrode and an external patch electrode (e.g., return electrodes 18, 20, and 21 (shown in FIG. 1)).

In this example, first waveform 802 includes a 3 μs positive pulse, followed by a 1 μs delay, followed by a 3 μs negative pulse. Second waveform 804 includes a 2 μs delay, followed by a 1 μs positive pulse, followed by a by a 1 μs delay, followed by a 1 μs negative pulse. In this embodiment, the pulses in first and second waveforms 802 and 804 have the same amplitude.

The shaft electrode only sees first waveform 802, and the patch electrode only sees second waveform 804. However, the tip electrode sees a combined waveform 806 that is the result of superimposing first waveform 802 onto second waveform 804. Here, as shown in FIG. 8, combined waveform 806 includes a 3 μs positive pulse, followed by a 1 μs delay, followed by a 3 μs negative pulse. Further, FIG. 8 also shows a current 808 seen by the tip electrode. Current 808 includes 2 μs at a first positive current level, followed by 1 μs at a second, higher positive current level, followed by 1 μs at a zero current level, followed by 1 μs at a first negative current level, followed by 2 μs at a second, lower negative current level.

As noted above, FIG. 8 is one example of superimposing a bipolar waveform (i.e., first waveform 802) with a monopolar waveform (i.e., second waveform 804). First waveform 802, by itself, results in relatively good energy delivery, but also limits current to the tip due to the surface area of the shaft electrodes. Second waveform 804, by itself, results in low or non-existent levels of muscle recruitment, while boosting current to the tip electrode. Accordingly, first and second waveforms 802 and 804 complement each other, and combining these two waveforms improving lesion efficiency and depth while reducing muscle recruitment.

Accordingly, as in this example embodiment, nesting multiple waveforms (e.g., monopolar and/or bipolar waveforms) between one or more active electrodes and multiple ground electrodes facilitates reducing or eliminating lesion generation at the ground electrodes while maintaining lesion quality at the active electrode(s), and also improves other characteristics of the therapy (e.g., reducing gas formation, patient movement, temperature increase, etc.). Those of skill in the art will appreciate that any suitable waveforms may be at least partially overlayed with one another. Further, a given waveform may oscillate between bipolar and monopolar energy delivery and/or between biphasic and monophasic energy delivery. Further, waveforms may be nested within each other as well, and may be fully overlapping, partially overlapping, have edge-to-edge spacing, or spaced with a gap between waveform deliveries. For fully or partially overlapping waveforms, one benefit is intensifying the energy delivery to the active electrode(s). For edge-to-edge spacing, one benefit is increasing energy delivery to the active electrode(s) while reducing energy delivery to any given return electrode. When spacing sequential waveforms with a gap, this facilitates reducing heating to the active electrode(s), while spreading energy across various return electrodes. In all cases, nested or sequential energy delivery to a plurality of returns may serve to reduce therapeutic, heating, and/or muscle recruitment to the return electrodes.

The active electrode may be, for example, a tip electrode, and the ground electrodes may be, for example, a shaft electrode, an external patch electrode, and/or an intra-vascular return electrode (i.e., an electrode located within the patient but on a separate catheter from the tip electrode). Those of skill in the art will appreciate that, in other embodiments, the active electrode(s) and ground electrodes may be any suitable electrodes.

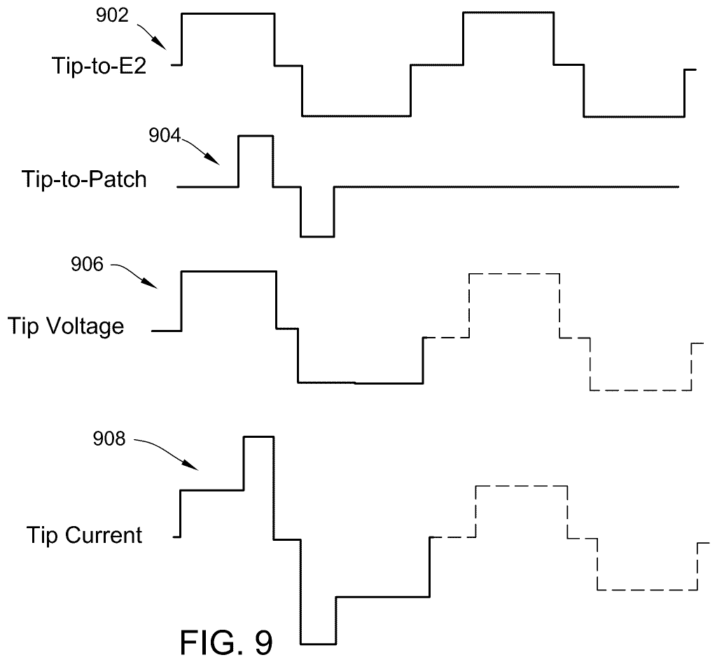

FIG. 9 is another example of overlaying two waveforms. In this example, a first waveform 902 is a bipolar waveform delivered between a tip electrode and a shaft electrode, E2. In contrast, a second waveform 904 is a monopolar waveform delivered between the tip electrode an external patch electrode (e.g., return electrodes 18, 20, and 21 (shown in FIG. 1)).

In this example, first waveform 902 includes a 3 μs positive pulse, followed by a 1 μs delay, followed by a 3 μs negative pulse, followed by a 2 μs delay, followed by a 3 μs positive pulse, followed by a 1 μs delay, followed by a 3 μs negative pulse. Here, the 2 μs delay is effectively a “pulse period” that defines a length of time until first waveform 902 repeats itself (e.g., with the initiation of another 3 μs positive pulse). Accordingly, although a 2 μs delay is shown and described, in other embodiments, the “pulse period” delay may be up to 100 milliseconds (ms). Further, those of skill in the art will appreciate that the various values (e.g., pulse lengths and amplitudes) shown and described herein are examples, and that other suitable values may be used without departing from the spirit and scope of the disclosure.

Second waveform 904 includes a 2 μs delay, followed by a 1 μs positive pulse, followed by a by a 1 μs delay, followed by a 1 μs negative pulse. In this embodiment, the pulses in first and second waveforms 902 and 904 have the same amplitude.

The shaft electrode only sees first waveform 902, and the patch electrode only sees second waveform 904. However, the tip electrode sees a combined waveform 906 that is the result of superimposing first waveform 902 onto second waveform 904. Here, as shown in FIG. 9, combined waveform 906 includes a 3 μs positive pulse, followed by a 1 μs delay, followed by a 3 μs negative pulse, followed by a 2 μs delay, followed by a 3 μs positive pulse, followed by a 1 μs delay, followed by a 3 μs negative pulse. Further, FIG. 9 also shows a current 908 seen by the tip electrode. Current 908 includes 2 μs at a first positive current level, followed by 1 μs at a second, higher positive current level, followed by 1 μs at a zero current level, followed by 1 μs at a first negative current level, followed by 2 μs at a second, lower negative current level, followed by 2 μs at the zero current level, followed by 3 μs at the first positive current level, followed by 1 μs at the zero current level, followed by 3 μs at the first negative current level.

Here, monopolar and bipolar pulses from first and second waveforms 902 and 904 only stack over roughly the first half of first waveform 902 (as the second half of second waveform 904 does not include any pulses).

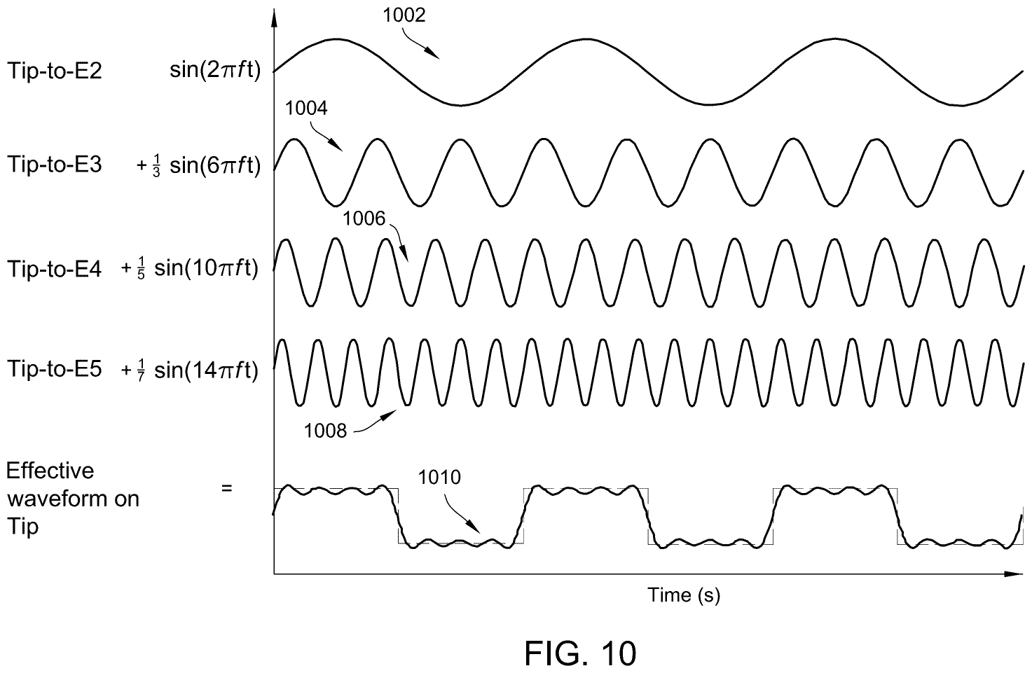

In some embodiments, waveforms having different pulse frequencies may be superimposed onto one another. In one example, multiple AC waveforms having different frequencies may be superimposed to create effective square wave pulses at the active electrode. FIG. 10 shows one example of this.

Specifically, FIG. 10 includes i) a first sinusoidal waveform 1002 delivered between a tip electrode and a first shaft electrode, E2, and having a first frequency, ii) a second sinusoidal waveform 1004 delivered between the tip electrode and a second shaft electrode, E3, and having a second frequency, iii) a third sinusoidal waveform 1006 delivered between the tip electrode and a third shaft electrode, E4, and having a third frequency, and iv) a fourth sinusoidal waveform 1008 delivered between the tip electrode and a fourth shaft electrode, E5. As used herein, a sinusoidal waveform may include a sine waveform, cosine waveform, and/or any other suitable waveform that may be added or subtracted to other waveforms to result in a desired pulse shape (e.g., a square pulse, a trapezoidal pulse, a triangular pulse, etc.).

Each shaft electrode only sees the associated sinusoidal waveform. However, the tip electrode sees a combined waveform 1010 that is the result of superimposing first sinusoidal waveform 1002, second sinusoidal waveform 1004, third sinusoidal waveform 1006, and fourth sinusoidal waveform 1008. As shown in FIG. 10, although waveforms 1002, 1004, 1006 and 1008 are all sinusoidal, resulting combined waveform 1010 effectively approximates a plurality of square wave pulses 1012.

Notably, the frequencies and amplitudes of the waveforms shown in FIG. 10 are merely examples, any suitable frequencies and amplitudes may be used to achieved desired pulse widths, etc. For example a 3 μs square wave pulse will require different sine components than a 5 μs square wave pulse.

Notably, sinusoidal waves are relatively easy to generate. Sinusoidal waves may also be less effective in causing electroporation, thus reducing collateral lesion formation on shaft electrodes. Also, sinusoidal waves generally reduce or eliminate muscle stimulation, and the resulting localized square wave seen by the catheter tip will stimulate less tissue overall,, reducing muscle contraction. Further, sinusoidal waves may be amplified using transformers, which reduces the need to use switching devices (e.g., IGBTs) to generate pulses. In addition, using a coil transformer to generate sine waves, high voltage pulses may be achieved using a lower voltage input.

The embodiment shown in FIG. 10 uses waveforms with different frequencies. Alternatively, sinusoidal waveforms with different phase shifts may be combined to generate a combined waveform that effectively approximates square wave pulses.

In other embodiments, effective square wave pulses could be achieved by combining other pulse shapes (e.g., triangular or trapezoidal pulse shapes). Using triangular pulse shapes may facilitate reducing sudden increase in membrane depolarization (due to the gradual rise and fall of the pulses), which in turn may facilitate reducing muscle recruitment.

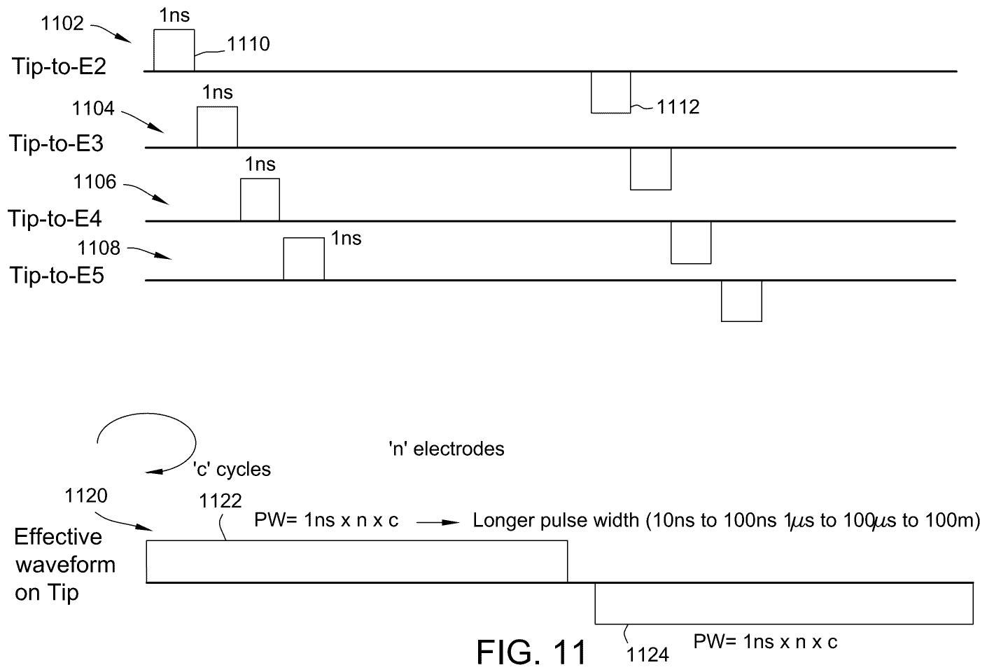

FIG. 11 is another example of superimposing or overlaying multiple waveforms. In this example, a first waveform 1102 is a bipolar waveform delivered between a tip electrode and a first shaft or spline electrode, E2. A second waveform 1104 is a bipolar waveform delivered between the tip electrode and a second shaft or spline electrode, E3. A third waveform 1106 is a bipolar waveform delivered between the tip electrode and a third shaft or spline electrode, E4. A fourth waveform 1108 is a bipolar waveform delivered between the tip electrode and a fourth shaft or spline electrode, E5. Additional, similar waveforms between the tip electrode and other shaft or spline electrodes may also be included (e.g., up to n waveforms). Further, a cycle of these waveforms may be repeated up to c times. Each waveform includes a single positive pulse 1110 having a pulse width of, for example, 1 ns, followed by a single negative pulse 1112 of, for example, 1 ns.

As shown in FIG. 11, when waveforms 1102, 1104, 1106, and 1108 are superimposed on one another, positive pulses 1110 and negative pulses 1112 are arranged such that a combined waveform 1120 includes an elongated positive pulse 1122 formed from positive pulses 1110 and an elongated negative pulse 1124 formed from negative pulses 1112. Elongated positive pulse 1122 and elongated negative pulse 1124 each have a pulse width equal to the number of individual waveforms (i.e., n), multiplied by the pulse width on each individual waveform (e.g., 1 ns), multiplied by the number of cycles (i.e., c). Alternatively, at least some of the pulses in one or more of the waveforms may have different pulse widths. Further, the pulse widths may be on the order of nanoseconds, microseconds, or any other suitable pulse width values. Notably, longer pulse widths will generally require use of less electrodes and fewer total pulses.

This concept may be applied to a relatively large number of individual waveforms (N), with each individual waveform having a pulse width of the desired total pulse width divided by N. For example, to obtain an effective pulse width of 3 μs at the tip electrode, six individual waveforms each having a pulse width of 500 ns could be applied (each individual waveform applied between the tip electrode and a different respective electrode). The electrodes could be included on any suitable type of catheter, so long as the proximity between the tip electrode and the other electrode for each individual waveform allows for a sufficiently effective electrical field for electroporation.

Alternatively, the individual waveforms may have different pulse widths. For example, a first waveform may have a pulse width of 1 μs, a second waveform may have a pulse width of 2 μs, and so on. This facilitates delivering shorter pulse widths between an external patch and a tip electrode than between a shaft electrode and the tip (e.g., to mitigate muscle stimulation associated with monopolar approaches).

In another example, a longer pulse width may be achieved at the tip using fewer total electrodes by cycling through the individual waveforms repeatedly (as illustrated in FIG. 11).

The use of relatively small pulse widths (e.g., on the order of 1 ns) in the individual waveforms reduces the time and possibility of cell excitation (thus potentially reducing muscle recruitment and arrythmias), and reduces or eliminates lesion generation at electrodes other than the active electrode while still generating a lesion at the active electrode (e.g., the tip electrode) because the electric field threshold for IRE is higher for smaller pulse widths.

In one embodiment, the “active” electrode is switched over time, resulting in multiple lesions or a larger lesion. For example, multiple individual waveforms may be applied between the tip electrode and various shaft or spline electrodes to generate an initial lesion. Subsequently, multiple individual waveforms may be applied between a particular shaft or spline electrodes and the tip electrode and other shaft or spline electrodes, creating an additional lesion at the particular shaft or spline electrode. If the tip electrode and the particular shaft or spline electrode are sufficiently proximate to one another, the initial lesion and the additional lesion may form a larger continuous lesion.

Of note, if the return electrodes are current limiting, multiple return electrodes may be ganged together for each individual waveform to achieve the desired current at the active electrode. For example, a first individual waveform may be applied between the tip electrode and the combination of E2 and E3, a second individual waveform may be applied between the tip electrode and the combination of E4 and E5, etc.

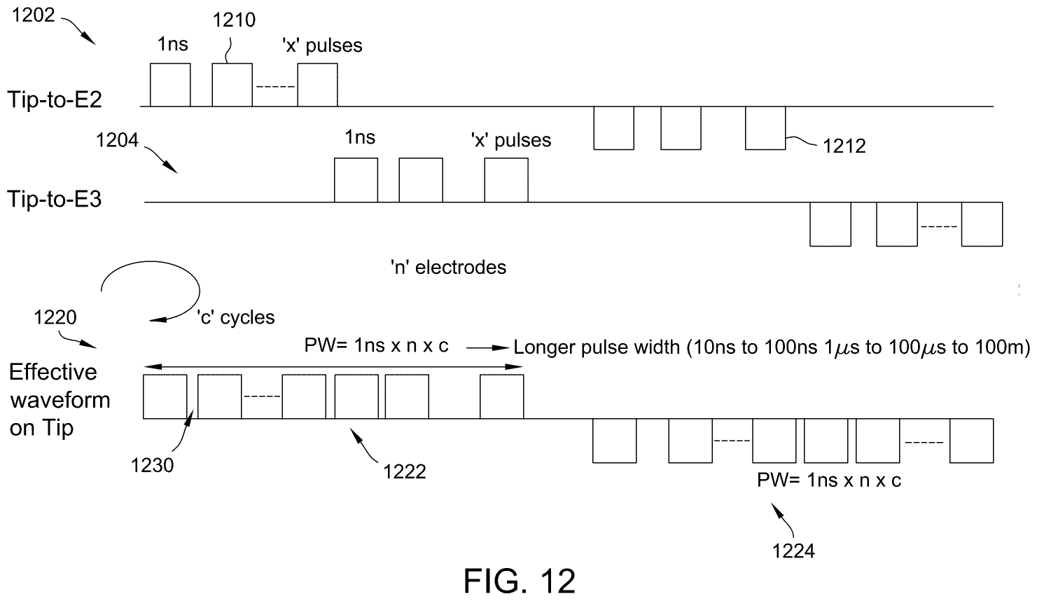

In another embodiment, shown in FIG. 12, each individual waveform may include multiple positive and negative pulses, instead of a single positive and negative pulse. Specifically, FIG. 12 shows a first waveform 1202 that is a bipolar waveform delivered between a tip electrode and a first shaft or spline electrode, E2, and a second waveform 1204 that is a bipolar waveform delivered between the tip electrode and a second shaft or spline electrode, E3. Additional, similar waveforms between the tip electrode and other shaft or spline electrodes may also be included (e.g., up to n waveforms). Further, a cycle of these waveforms may be repeated up to c times. Each waveform includes multiple single positive pulses 1210 having a pulse width of 1 ns, followed multiple negative pulses 1212 of 1 ns. Alternatively, at least some of the pulses in one or more of the waveforms may have different pulse widths. Further, the pulse widths may be on the order of nanoseconds, microseconds, or any other suitable pulse width values. Notably, longer pulse widths will generally require fewer total pulses (and may require using fewer electrodes).

As shown in FIG. 12, when waveforms 1202 and 1204 are superimposed on one another, positive pulses 1210 and negative pulses 1212 are arranged such that a combined waveform 1220 includes an effective elongated positive pulse 1222 (formed of multiple, closely spaced positive pulses) and an effective elongated negative pulse 1224 (formed of multiple, closely spaced negative pulses). Effective elongated pulses 1222 and 1224 may have an effective pulse width on the order of 10 ns, on the order of 100 ns, on the order of 1 μs, on the order of 100 μs, on the order of 1 ms, or on the order of 100 ms.

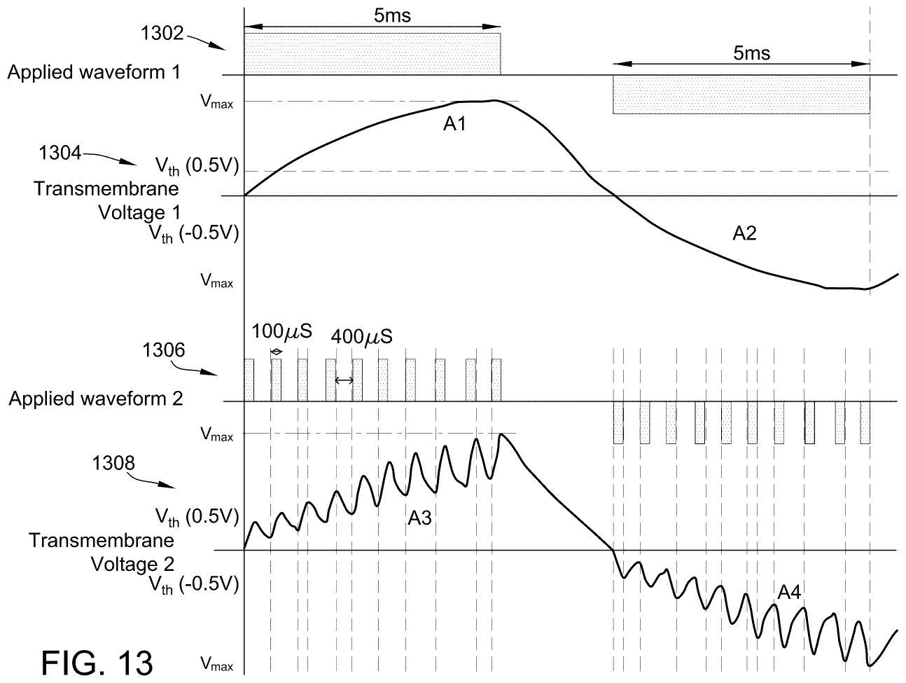

Notably, gaps 1230 between the individual pulses constituting elongated pulses 1222 and 1224 generally do not negatively impact the therapeutic effectiveness of elongated pulses 1222 and 1224 due to the relatively slow decay of the transmembrane potential. In fact, these gaps 1230 facilitate reducing heating, microbubble formation, and arrhythmias, as the smaller pulses have a higher threshold for tissue excitation. FIG. 13 demonstrates this concept.

Specifically, FIG. 13 includes a first applied waveform 1302 including a 5 ms positive pulse followed by a 5 ms negative pulse. First applied waveform 1302 results in first transmembrane potential 1304. In contrast, a second applied waveform 1306 includes a plurality of smaller closely spaced positive pulses and closely spaced negative pulses, which results in second transmembrane potential 1308.

As shown in FIG. 13, the small pulses of second applied waveform 1306 result in second transmembrane potential 1308 having a sawtooth-like shape. However, second transmembrane potential 1308 still maintains a similar area under the curve as first transmembrane potential 1304, which is sufficient for effective electroporation. That is the area under first transmembrane potential 1304 (A1+A2) is substantially equal to the area under second transmembrane potential 1308 (A3+A4).

In second applied waveform 1306, the membrane potential discharges naturally during the gaps between pulses. This allows the membrane potential to stay above the electroporation threshold without requiring constant energy delivery. This concept could be extended to tens or hundreds of nanosecond pulses with optimized gaps between pulses to generate effective electroporation. Depending upon the gaps between pulses, it is possible to optimize an off time that achieves a maximum area above the electroporation threshold while minimizing a number of pulses. The optimized off time would also depend on the pulse width. For example, a nanosecond pulse (as compared to a microsecond pulse) will charge the membrane less and have a faster discharge.

Although in at least some of the embodiments described herein, the superimposed waveforms share at least some zero voltage segments, this is not a requirement. Further, monophasic and biphasic waveforms, as well as monopolar and bipolar waveforms may be combined with another, as described herein. Those of skill the art will appreciate that many different combinations of superimposed waveforms are within the scope of the present disclosure.

The systems and methods described herein are directed to electroporation. An electroporation system includes a catheter including a plurality of electrodes, and a pulse generator coupled to the catheter. The pulse generator is configured to generate a first waveform to be delivered between at least one active electrode on the catheter and at least one first return electrode, generate a second waveform to be delivered between the at least one active electrode and at least one second return electrode, and cause delivery of the first and second waveforms over the same time period such that at least the first and second waveforms are superimposed onto one another to create a combined waveform at the at least one active electrode.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. An electroporation system comprising:
a catheter comprising a plurality of electrodes; and
a pulse generator coupled to the catheter, the pulse generator configured to:
generate a first waveform to be delivered between at least one active electrode on the catheter and at least one first return electrode;
generate a second waveform to be delivered between the at least one active electrode and at least one second return electrode; and
cause delivery of the first and second waveforms over the same time period such that at least the first and second waveforms are superimposed onto one another to create a combined waveform at the at least one active electrode.

2. The electroporation system in accordance with claim 1, wherein at least one of the at least one first return electrode is an electrode on the catheter.

3. The electroporation system in accordance with claim 1, wherein at least one of the at least one first return electrode is an external patch electrode.

4. The electroporation system in accordance with claim 1, wherein at least one of the at least one first return electrode is an intra-vascular electrode located on an additional catheter.

5. The electroporation system in accordance with claim 1, wherein at least one of the first and second waveforms includes non-square wave pulses.

6. The electroporation system in accordance with claim 1, wherein the first waveform is a bipolar waveform, and wherein the second waveform is a monopolar waveform.

7. The electroporation system in accordance with claim 1, wherein the first waveform is a biphasic waveform, and wherein the second waveform is a monophasic waveform.

8. The electroporation system in accordance with claim 1, wherein the first and second waveforms are both bipolar waveforms.

9. The electroporation system in accordance with claim 1, wherein the catheter is at least one of a loop catheter assembly, a linear catheter assembly, a basket catheter assembly, a grid catheter assembly, a balloon catheter assembly, a sheath, and a sleeve.

10. The electroporation system in accordance with claim 1, wherein the first and second waveforms are both sinusoidal waveforms, and wherein the combined waveform generated from at least the first and second waveforms includes a plurality of effective square wave pulses.

11. The electroporation system in accordance with claim 1, wherein the combined waveform includes at least one effective pulse that has a longer effective pulse width than any of the pulses in the first and second waveforms.

12. The electroporation system in accordance with claim 11, wherein the at least one effective pulse is a continuous pulse.

13. The electroporation system in accordance with claim 11, wherein the at least one effective pulse includes a plurality of closely spaced smaller pulses.

14. A pulse generator for use with an electroporation system, the pulse generator configured to be coupled to a catheter including a plurality of electrodes and configured to:
generate a first waveform to be delivered between at least one active electrode on the catheter and at least one first return electrode;
generate a second waveform to be delivered between the at least one active electrode and at least one second return electrode; and
cause delivery of the first and second waveforms over the same time period such that at least the first and second waveforms are superimposed onto one another to create a combined waveform at the at least one active electrode.

15. The pulse generator in accordance with claim 14, wherein the first waveform is a bipolar waveform, and wherein the second waveform is a monopolar waveform.

16. The pulse generator in accordance with claim 14, wherein the first waveform is a biphasic waveform, and wherein the second waveform is a monophasic waveform.

17. A method for electroporation therapy, the method comprising:
generating, using a pulse generator, a first waveform to be delivered between at least one an active electrode on a catheter and at least one first return electrode;
generating a second waveform to be delivered between the at least one active electrode and at least one second return electrode; and
causing delivery of the first and second waveforms over the same time period such that at least the first and second waveforms are superimposed onto one another to create a combined waveform at the at least one active electrode.

18. The method in accordance with claim 17, wherein at least one of the at least one first return electrode is an electrode on the catheter.

19. The method in accordance with claim 17, wherein at least one of the at least one first return electrode is an external patch electrode.

20. The method in accordance with claim 17, wherein the combined waveform includes at least one effective pulse that has a longer effective pulse width than any of the pulses in the first and second waveforms.
